(19) United States
(12) Patent Application Publication
(10) Pub. No.: US 2021/0007878 A1
(43) Pub. Date: Jan. 14, 2021

(54) ANTI-FATIGUE FOOT PAD MANUFACTURING METHOD

(71) Applicant: XIAMEN TUOQIANG INDUSTRIAL & TRADING CO., LTD., Xiamen City (CN)

(72) Inventor: Xurong ZHONG, XIAMEN CITY (CN)

(21) Appl. No.: 17/033,084

(22) Filed: Sep. 25, 2020

Related U.S. Application Data
(62) Division of application No. 15/893,251, filed on Feb. 9, 2018.

(30) Foreign Application Priority Data
Dec. 25, 2017 (CN) 201711416796.9

(51) Int. Cl. A61F 5/34; A61H 15/00; A61H 1/02

(52) U.S. Cl. CPC A61F 5/34; A61H 15/00; A61H 1/0274; A61H 2001/0248; A61H 2205/12; A61H 2201/0103; A61H 1/0237

(57) ABSTRACT

An anti-fatigue foot pad manufacturing method. According to the anti-fatigue foot pad manufacturing method, a protective layer is formed in a cavity of a mold firstly, and then massage blocks and a foamed elastic body are fixed by the protective layer, so that the massage blocks are mounted and supported independently, and the technical defect that in the prior art, all massage blocks/protrusions have to be fixed to a hard support plate is overcome.

### CROSS REFERENCE TO RELATED APPLICATIONS

This application is a Divisional of co-pending application Ser. No. 15/893,251, filed on 9 Feb. 2019, for which priority is claimed under 35 U.S.C. § 120; and this application claims priority of Application No. 201711416796.9 filed in CHINA on 25 Dec. 2017 under 35 U.S.C. § 119; the entire contents of all of which are hereby incorporated by reference.

### BACKGROUND OF THE INVENTION

### Technical Field

The invention belongs to the technical field of fitness equipment, and particularly relates to an anti-fatigue foot pad manufacturing method and an anti-fatigue foot pad.

### Description of Related Art

Foot pads with a massage function are fitness/health-care equipment which can relieve foot fatigue in life and work by massaging foot muscles.

In the prior art, a foot pad is mainly provided with a massage area where the foot can be placed, certain protrusions or moving mechanisms used for massage are arranged in the massage area, and although the foot pad has a massage/health-care function, the massage area generally has the following technical problems:

(1) the protrusions are arranged in the massage area and formed on an elastic supporting mechanism, the protrusions and the elastic supporting mechanism are made of similar or identical materials, the self strength of the protrusions is not high enough, and consequentially the massage function cannot be well achieved;

(2) a supporting plate is arranged in the massage area, the protrusions are arranged on the massage plate, and the massage plate is then supported by elastic materials; in this structure, most protrusions are integrally fixed together, a single protrusion or several local protrusions cannot elastically float independently, and consequentially, the massage hardness is large, and the massage area cannot apply force to or make contact with the foot for a long time.

Therefore, to solve the problem that all massage protrusions have to be integrally combined or formed on an elastic body, a massage foot pad which allows hard massage protrusions to be supported and bounced independently to achieve a massage function is urgently needed, and a novel massage pad production technique is also urgently needed.

### BRIEF SUMMARY OF THE INVENTION

To solve the problem that massage protrusions of an existing massage foot pad cannot be supported independently, the invention aims to provide an anti-fatigue foot pad manufacturing method.

The specific scheme is as follows: an anti-fatigue foot pad manufacturing method comprises the following steps:

Step 1, preparation of a protective layer, specifically, a mold is provided, and pits are formed in a cavity of the mold; a film forming agent is evenly sprayed onto the inner surface of the cavity of the mold, and the protective layer is prepared after the film forming agent is coagulated;

Step 2, fixing of massage blocks, specifically, hard massage blocks are arranged on the protective layer formed in Step 1 and located at positions corresponding to the pits, and thus the massage blocks are fixed;

Step 3, foaming of an elastic body, specifically, foam materials are injected into the cavity in Step 2 and foamed and cured, so that the elastic body is prepared.

Furthermore, Step 1 preferably comprises the following Sub-steps:

Sub-step 11, heating the mold and spraying a releasing agent into the cavity of the mold;

Sub-step 12, brushing the cavity of the mold with dry air;

Sub-step 13, spraying the film forming agent into the cavity.

Furthermore, in Step 1, the mold is maintained at the temperature from 50 DEG to 70 DEG C. after being heated, and the film forming agent is the mixed liquor of polyether polyol and isocyanate.

Furthermore, the massage blocks are one type or the combination of several types of steel balls, pebbles, plastic blocks, and inflated balls.

Furthermore, in Step 3, the foam materials are polyurethane; after polyurethane is injected into the cavity, an upper mold body and a lower mold are assembled together to seal the cavity, and thus the polyurethane is foamed and cured.

The invention further provides an anti-fatigue foot pad which comprises a protective layer, massage blocks and an elastic body, wherein the elastic body is arranged on the inner surface of the protective layer, the massage blocks are arranged between the protective layer and the elastic body, and the elastic body is made of foam materials with the shape memory characteristic.

Furthermore, the massage blocks are inflated balls, the bottom of each inflated ball is arranged in the elastic body, and the rest part of each inflated ball stretches out of the elastic body and is wrapped in the protective layer.

Furthermore, the inflated balls are netted balls, and the depth of the portion, located in the elastic body, of each netted ball is one third of the diameter of the netted ball.

Beneficial effects: according to the anti-fatigue foot pad manufacturing method, firstly, the protective layer is formed in the cavity of the mold, and then the massage blocks and the foamed elastic body are fixed by the protective layer, so that the massage blocks are mounted and supported independently, and the technical defect that in the prior art, all massage blocks/protrusions have to be fixed to a hard support plate is overcome; secondly, the protective layer is supported by the coagulated film forming agent, and the foam materials are arranged in the protective layer, so that the problems that in the prior art, foam materials are poor in surface quality, and bubbles, scorches or notches are likely to be generated on the surfaces of the foam materials are solved; the hardness of the protective layer can affect the comfort level of the foot pad, and the technical formula can be adjusted to further adjust the hardness of the elastic body, so that the comfort level desired by users can be achieved, the surface of the protective layer is free of bubble holes, and thus the foot pad is convenient to clean; in addition, the cavity of the mold is provided with the pits, the shape and size of the pits are matched with those of the corresponding massage blocks, the massage blocks are fixed in the foaming process of the elastic body by means of the viscosity of the protective layer and the shape cooperation with the pits, and the technical problem that the positions of independent massage blocks are difficult to fix is solved.

The anti-fatigue foot pad of the invention is provided with the smooth and wearable protective layer, the independent massage blocks and the elastic body, and has a massage function achieved through hard massage protrusions which are supported and bounced independently.

### BRIEF DESCRIPTION OF THE SEVERAL VIEWS OF THE DRAWINGS

FIG. 1 is a structural diagram of an anti-fatigue foot pad in a second embodiment of the invention; and

FIG. 2 is a sectional view of FIG. 1.

### DETAILED DESCRIPTION OF THE INVENTION

The drawings are provided for a further description of the embodiments of the invention. These drawings are part of the content of the invention, are mainly used for illustrating the embodiments and can also be used for explaining the operating principle of the embodiments in cooperation with relevant content in the description. By referring to the content, those ordinarily skilled in the field can understand other possible embodiments and the advantages of the invention. Components in the drawings are not drawn in proportion, and similar component symbols are generally used for representing similar components.

A further description of the invention is given with accompanying drawings and specific embodiments as follows.

### First Embodiment

The inventor finds through study that, in the prior art, all massage blocks have to be formed on one base plate so as to be mounted and fixed, and then the base plate is mounted on an elastic material.

The main technical defects of the prior art: (1) massage blocks cannot be fixed independently, and by adoption of the elastic material, the positions of the massage blocks are extremely likely to be changed in the foam forming process; (2) if the massage blocks are directly fixed in a cavity of a mold, the massage blocks are likely to fall from the elastic material or need to be bonded and fixed later, the bonding surface is extremely likely to fall and fail when users tread on the bonding surface, and thus the product quality is poor; a structure internally provided with independent massage blocks cannot be produced actually.

The embodiment of the invention provides a manufacturing method of a non-bonded anti-fatigue foot pad with massage blocks fixed independently, and the manufacturing method comprises the following steps:

Step 1, preparation of a protective layer, specifically, in the Step, a mold is provided firstly, the mold comprises a lower mold body provided with a cavity, and a planar upper mold body, an opening of the cavity is vertically placed upwards, and the bottom surface of the cavity of the mold is provided with a plurality of pits used for accommodating massage blocks;

Specifically, Step 1 comprises the following Sub-steps:

Sub-step 11, heating the mold, and preferably maintaining the mold at a constant temperature which ranges from 50 DEG C. to 70 DEG C. and depends on parameter requirements of a film foaming agent after the mold is heated; afterwards, cleaning the cavity, and spraying a releasing agent into the cavity of the mold, so that the releasing agent is attached to the inner surface of the cavity of the mold after being heated;

Sub-step 12, brushing the cavity of the mold with high-pressure dry air blown out by an air gun, so that non-solidified releasing agent residues in the liquid stage are blown away;

Sub-step 13, evenly spraying the film agent into the cavity, wherein the film forming agent is preferably the mixed liquor of polyether polyol and isocyanate, and the mixed liquor is coagulated and solidified under the effect of the self temperature of the mold, and thus the protective layer is formed; the Sub-step can be executed repeatedly, so that the thickness of the protective layer is controlled.

Step 2, fixing of massage bocks, specifically, hard massage blocks are arranged on the inner side of the protective layer formed in Step 1 and located at positions corresponding to the pits in the bottom surface of the cavity, and then the massage blocks are fixed by means of the viscidity of the protective layer and dimensional coordination of the massage blocks and the pits; the massage blocks can be one type or the combination of several types of steel balls, pebbles, plastic blocks, and inflated balls such as netted balls.

Step 3, foaming of an elastic body, specifically, foam materials are injected into the cavity in Step 2, and in the embodiment, the foam materials are polyurethane; the foam materials are foamed and cured, and thus the elastic body is prepared.

Furthermore, in Step 3, after polyurethane is injected into the cavity, the upper mold body is assembled on the lower mold body in a covering mode, so that the cavity is sealed, and the polyurethane is foamed and cured.

Step 4, mold opening, specifically, the anti-fatigue foot pad in the cavity is taken out.

### Second Embodiment

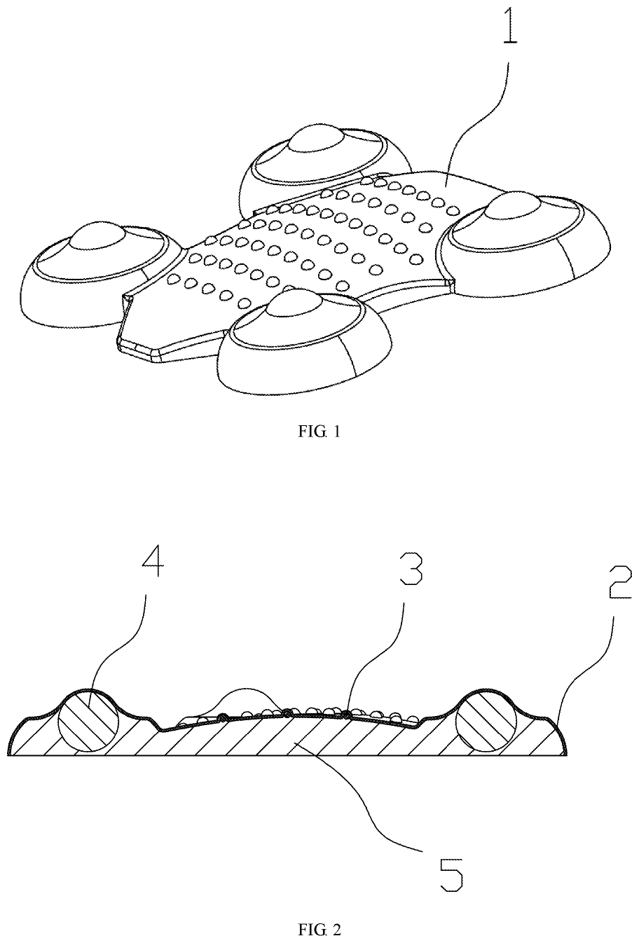

As is shown in FIG. 1 and FIG. 2, the second embodiment of the invention provides an anti-fatigue foot pad 1 which sequentially comprises, from outside to inside, a protective layer 2, first massage blocks 3, second massage blocks 4 and an elastic body 5.

In the embodiment, the protective layer 2 is formed by a coagulated and solidified film forming agent, and the elastic body 5 is arranged on the inner surface of the protective layer and is made of shape memory materials, namely polyurethane, formed on the inner side of the protective layer in a foamed mode.

In the embodiment, the first massage blocks 3 are steel balls arranged between the protective layer 2 and the elastic body 5, and the first massage blocks 3 are arranged in the middle of the upper end face of the elastic body 5 to serve as a pedal part.

In the embodiment, the second massage blocks 4 are inflated balls and preferably are netted balls, and the second massage blocks 4 are arranged between the protective layer 2 and the elastic body 5; in the embodiment, the elastic body 5 is approximately a cuboid, the number of the second massage blocks 4 is four, and the four second massage blocks 4 are separately arranged at the four top corners on the upper end face of the elastic body; as is shown in the FIGs, the bottom of each inflated ball is wrapped in the elastic body 5, the top of each inflated ball stretches out of the elastic body 5 and is wrapped in the protective layer 2, and then the top of each second massage block 4 stretches out of the upper end face of the elastic body 5, so that a stretchy support part used for supporting the foot arch is formed.

In can be understood that in the embodiment, the depth of the portion, embedded in the elastic body 5, of each second massage block 4 is two thirds of the diameter of the second massage block 4, namely one third of each second massage block 4 is embedded in the protective layer 2, and thus the second massage blocks 4 are fixed in the foaming process; in other embodiments, the inflated balls are netted balls, the depth of the portion, embedded in the elastic body, of each netted ball is one third of the diameter of the netted ball, then the second massage blocks 4 can be elastically supported, and the protective layer 2 can be prevented from falling.

Although the invention is specifically illustrated and introduced with the preferred embodiments, those skilled in the field should understand that various changes of the invention made in form and in detail without deviating from the spirit and scope defined by the claims are all within the protection scope of the invention.

What is claimed is:

1. An anti-fatigue foot pad manufacturing method, wherein comprising the following steps:
Step 1, preparation of a protective layer, specifically, a mold is provided, and pits are formed in a cavity of the mold; a film forming agent is evenly sprayed onto the inner surface of the cavity of the mold, and the protective layer is prepared after the film forming agent is coagulated;
Step 2, fixing of massage blocks, specifically, hard massage blocks are arranged on the protective layer formed in Step 1 and located at positions corresponding to the pits, and thus the massage blocks are fixed;
Step 3, foaming of an elastic body, specifically, foam materials are injected into the cavity in Step 2 and foamed and cured, so that the elastic body is prepared.

2. The anti-fatigue foot pad manufacturing method according to claim 1, wherein Step 1 comprises the following Sub-steps:
Sub-step 11, heating the mold and spraying a releasing agent into the cavity of the mold;
Sub-step 12, brushing the cavity of the mold with dry air;
Sub-step 13, spraying the film forming agent into the cavity.

3. The anti-fatigue foot pad manufacturing method according to claim 2, wherein the mold is maintained at the temperature from 50 DEG C. to 70 DEG C. after being heated, and the film forming agent is the mixed liquor of polyether polyol and isocyanate.

4. The anti-fatigue foot pad manufacturing method according to claim 1, wherein the massage blocks are one type or the combination of several types of steel balls, pebbles, plastic blocks, and inflated balls.

5. The anti-fatigue foot pad manufacturing method according to claim 1, wherein the foam materials are polyurethane; after polyurethane is injected into the cavity, an upper mold body and a lower mold are assembled together to seal the cavity, and thus the polyurethane is foamed and cured.
